(19) United States
(12) Patent Application Publication
(10) Pub. No.: US 2015/0218501 A1
(43) Pub. Date: Aug. 6, 2015

(54) DISPOSABLE BOTTLE REACTOR TANK

(71) Applicant: BAYER TECHNOLOGY SERVICES GMBH, Leverkusen (DE)

(72) Inventors: Joerg Kauling, Bergisch Gladbach (DE); Annette Waldhelm, Odenthal (DE); Helmut Brod, Koln (DE); Verena Herrmann, Koln (DE); Stefanie Jacob, Koln (DE)

(73) Assignee: BAYER TECHNOLOGY SERVICES GMBH, Leverkusen (DE)

(21) Appl. No.: 14/429,055

(22) PCT Filed: Sep. 13, 2013
(86) PCT No.: PCT/EP2013/069024

§ 371 Date: Mar. 18, 2015

(30) Foreign Application Priority Data
Sep. 18, 2012 (EP) 12006536.2

(51) Int. Cl. C12M 1/24; C12M 1/12; C12M 3/04; C12M 1/00

(52) U.S. Cl. CPC C12M 23/08; C12M 23/38; C12M 23/28; C12M 37/00; C12M 37/02; C12M 29/20; C12M 23/34; C12M 29/00; C12M 27/10

(57) ABSTRACT

The invention relates to a reactor tank designed as a disposable element having a cover and/or sensor patches readable opto-electronically fixed in the interior, a reactor comprising the reactor tank and reactor tank receiving peripherals, the same comprising a reactor tank holder and optionally an opto-electronic measuring system for reading sensor patches, wherein the reactor tank holder is coupled to a drive unit for generating a rotating-oscillation motion of the reactor tank around the central vertical axis thereof, and also the use of this device for culturing cells and/or microorganisms.

The invention relates to a reactor tank designed as a disposable element having a cover and/or sensor patches readable opto-electronically fixed in the interior, a reactor comprising the reactor tank and reactor tank receiving peripherals comprising a reactor tank holder and optionally an opto-electronic measuring system for reading the sensor patches, wherein the reactor tank holder is coupled to a drive unit for generating a rotating-oscillation motion of the reactor tank around the central vertical axis thereof, and also the use of this device for culturing cells and/or microorganisms.

In the case of the highly regulated pharmaceutical production, a great expenditure in terms of time, technical expenditure and expenditure on staff is accounted for by the provision of cleaned and sterilized bioreactors. In order to avoid reliably cross contamination during a product change in a multi-purpose plant or between two product batches, in addition to the cleaning, a highly complex cleaning validation is required which, in the case of a process adaption, may need to be repeated.

This applies not only to upstream processing, USP, as to say the production biological products in fermenters, but also to downstream processing, DSP, that is to say purification of the fermentation products.

In USP and DSP, pressure vessels are frequently used as agitation and reaction systems. Especially in the case of fermentation, an aseptic environment is essential for successful culturing. For sterilization of batch or fed-batch fermenters, generally the steam-in-place (SIP) technique is used. In order in the case of a continuous process procedure to ensure sufficient long term sterility, the autoclaving technique is also utilized which, however, requires laborious transport of the reactors to the autoclave, and is only useable at comparative small reactor scales. The risk of contamination during fermentation is particularly critical during sampling and at moving stirrer shafts. The latter are generally equipped with complex sealing systems (e.g.: sliding-ring seals). Technologies which manage without such penetrations of the fermentation casing are preferred because of their greater process robustness.

The down time of standard reactors caused by the preparation procedures can be in the order of magnitude of the reactor availability, in particular in the case of short utilization periods and frequent product change. The process steps affected are, in USP of the biotechnological production, e.g. the steps of media production and fermentation, and in DSP solubilisation, freezing, thawing, pH adjustment, precipitation, crystallization, buffer change and virus inactivation.

In order to meet the requirement of rapid and flexible re-charging of the production plant, while ensuring maximum cleanness and sterility, designs of disposable reactors are enjoying constantly increasing interest on the market.

WO 2007/121958 A1 and WO2010/127689 describe such a disposable reactor for culturing cells and microorganisms. In one preferred embodiment it consists of a stable, preferably multilayer, polymer material pouch. The deformable disposable reactor is received by a container which supports it. In this process it is preferably introduced into the container from the front. The container is connected to a drive unit. By way of the drive unit, the container including the disposable reactor is put into a rotating-oscillating motion about a stationary, preferably vertical, axis of the container. By way of an square design shape of the disposable reactor and/or internals in the disposable reactor, in the case of the oscillating-rotating motion, a high introduction of work into the reactor contents can be achieved, so the disposable reactor can be used as a fermenter with surface gas treatment for culturing cells and microorganisms. The internals for supplying and monitoring reactor are mounted at the side at the bottom of the reactor via a connecting plate. These reactors are predominantly used at reactor volumes of more than 10 L.

For smaller reactor volumes, the production of a reactor pouch including appropriate container is too complex.

The challenge in small disposable reactors is to achieve the sensor technology, the mixing technology, the temperature control and supplying of the reactor in a form that is as compact and inexpensive as possible.

Small agitated disposable reactors are known from the prior art.

Sartorius Stedim Biotech, in its Universel® SU (http://www.sartorius-stedim.com/Biotechnology/Fermentation_Technologies/Reusable_Bioreactors/Data_Sheets/Data_UniVessel_SU_SBI2033-e.pdf), offers an agitated disposable reactor in which the reactor tank is cylindrical. The disposable reactor, for the mixing, possesses an agitator, and for the gas supply from below, possesses an L-sparger below the agitator. Via the lid, the agitator drive is ensured by a top-driven drive axle, the sensor technology (temperature, pH (chemistry), oxygen (chemistry)), the gas supply and gas disposal for the gas space, and further supply and sampling via conduits. The lid is fastened to the reactor tank by a clamp connection, and is sealed in a sterile manner against the reactor tank by O ring. The agitator drive is sealed with 2 lip seals. The sensor technology for monitoring pH and oxygen content can also be achieved by means of optoelectronic sensor patches at the bottom of the reactor tank. For operation, the reactor tank is positioned fixed in a special container, wherein this container possesses a holder ring and a foot having an optoelectronic sensor system for reading the sensor patches.

The disadvantage of this and/or similar reactor systems available on the market is that these stirred systems require moving internals and also a complex sterile sealing system in the lid, and, in view of the high shear forces are less suitable for the culture of very sensitive cells, such as e.g. stem cells.

Proceeding from the prior art, the object in question is to provide a low-shear system for carrying out processes having high requirements of cleanliness and sterility which reduces the expenditure in terms of time, equipment and staff on the provision of cleaned and sterilized components. The system shall be useable for process volumes from 10 mL to 20 L, in particular 50 mL to 10 L, and particularly preferably 250 mL to 3 L working volume. It shall meet the high requirements of the pharmaceutical industry, be simple and intuitive to handle and be inexpensive. It shall reduce safety risks due to the escape of substances from the process chamber to a minimum. It shall permit sufficient mixing of the reactor contents, be suitable for the culture of microorganisms and cell cultures and, in the process, ensure sufficient supply and disposal of the culture medium with liquid nutrient medium and in particular gaseous substances. It shall be just as suitable for process development as for the production of cell products, in particular cell products such as, e.g., human or animal body cells: stem cells, blood cells, leucocytes such as, e.g., natural killer cells (NK cells), tissue cells or pharmaceutical active ingredients such as, e.g., monoclonal antibodies, proteins, enzymes in bioreactors.

According to the invention this object is achieved by the use of a dimensionally stable, angular plastics bottle for delimiting the reactor interior, wherein the plastics bottle has a bottom, walls, an interior and at least one access to the interior, and preferably a pyramidal inwards-dished bottom, a wide neck and/or one or more sensor patches mounted in the lower region of the bottle at a site defined by coordinates.

The present invention first therefore relates to the use of a dimensionally stable, angular plastics bottle as bioreactor tank for the culture of cells, in particular sensitive cells and cells growing on (micro)supports, such as, e.g., stem cells, blood cells or tissue cells, wherein the plastics bottle has a bottom, walls, an interior and at least one closable access to the interior, in particular a bottleneck. Usually, in the interior of the plastics bottle, one or more sensor patches are mounted on one or more walls in the lower region, at a site defined by coordinates.

The present invention further relates to a reactor tank comprising a dimensionally stable angular plastics bottle which has a bottom, walls, an interior and at least one closable access to the interior, comprising at least one bottleneck, in particular closable by a lid, and/or at least one passage, and wherein one or more sensor patches are mounted in the interior, on one or more walls in the lower region of the plastics bottle, at a site defined by coordinates. Preferably, passages are accommodated in the lid.

Support-fixed sensor patches made up of fluorescent coloured layers are available on the market (e.g. from Presens, YSI) which can be affixed, e.g., to a bottle wall. Usually, at least one pH sensor patch and one oxygen sensor patch are used.

Alternatively, the reactor tank or bioreactor has passages for electrochemical sensors, preferably disposable sensors, e.g. according to US 20120067724 A1, on a bottle wall or in the lid, preferably in the lid.

In order that the reactor can meet the sterility requirements of the pharmaceutical industry, the plastics bottle is usually produced from a gamma-sterilizable plastics material. The reactor tank according to the invention is preferably made from a single- or multilayer transparent polymer material which permits a view into the reactor tank during operations.

Plastics or glass are relatively inexpensive materials which may also be processed relatively inexpensively. The disposal of the used reactor tank and the use of a new disposable reactor tank are thus more economical than cleaning used reactor tanks, in particular, since when a new disposable reactor tank is used, complex cleaning and cleaning validation are omitted. The reactor tank according to the invention is produced or cleaned in a cleanroom and is preferably sterile-packed.

The reactor tank according to the invention is dimensionally stable. Suitable materials or material combinations for the reactor tank according to the invention are all cell biological compatible materials known to those skilled in the art, in particular glass, polyethylene, polypropylene, polyetherketone (PEEK), PVC, polyethylene terephthalate and polycarbonate. Wall thicknesses of 0.1 mm-5 mm are preferred, and of 0.5-2 mm are particularly preferred.

The bottle materials are usually brought into the desired form by means of stretch blow moulding methods known from the prior art.

The cross section of the reactor tank or of the plastics bottle preferably has the shape of an n-gon where n is in the range from 3 to 12, preferably in the range from 3 to 6, very particularly preferably in the range 3 to 4, most preferably, n is equal to 4.

Preferably, the side walls of the reactor tank according to the invention or the plastics bottle are formed at least in part as flat surfaces which meet at an angle of 45° to 120°. Preferably, the side walls of the reactor form a polyhedron, wherein the bottleneck is mounted on one of the surfaces.

Preferably, the reactor tank or the plastics bottle is cuboidal with edge lengths H, b and c, wherein H is the height, b is the width and c is the depth of the plastics bottle and b≦c≦H. The wide neck is typically mounted on one of the small surfaces and the surface opposite it serves as bottom of the reactor tank. The reactor tank according to the invention or the plastic bottle have a ratio of bottle height H to maximum width b and depth c in the range from 0.5 to 4, preferably 1 to 3, particularly preferably 1.5 to 2.5. In the preferred embodiment, the reactor tank has a square bottle cross section edge length a=c=D.

For better mixing of the reactor and reduced starting volume, the reactor tank and/or the plastics bottle usually has an inwards-dished bottom. For the configuration of the bottom, the teaching of WO 2010/127689 is incorporated by reference. The bottom has, in particular, the shape of an inwards-directed tetrahedron, an inwards-directed pyramid, the shape of a paraboloid or a bell shape. Particularly preferably, the bottom is formed pyramidally. The height h_{w} of the dishing is in the range of 0.01 times to 1 times the circular equivalent diameter Dₖ of the bottom cross section. Preferably, the height h_{w} of the dishing to the circle-equivalent diameter Dₖ is in the range from 3% to 100%, particularly preferably in the range from 5% to 30%, and very particularly preferably in the range from 10% to 20%.

The reactor tank according to the invention can be heated and/or cooled via the outer walls thereof. In a preferred embodiment, on the outside of the bottom of the plastics bottle or the reactor tank, a disposable heating mat is applied with which, owing to the positive connection of heating surface and shell surface, very efficient heat transport can be achieved. In this manner, the heating surface can be reduced to the bottom surface. For this purpose, this heating mat is usually adhesively connected to the outside of the bottom. Generally, the reactor tank does not need additional cooling, since switching off the heating mat in reactors having a small volume and thus high specific heat exchange surface area, leads to sufficiently rapid cooling. Additional cooling would be applicable if required, e.g., in the case of microbial applications at relatively low fermentation temperature and relatively high heat of respiration, by mounting Peltier elements to the side surfaces of the reactor tank or of the tank holder.

The reactor tank according to the invention is preferably a chamber that can be sealed off from the outside for carrying out chemical, biological, biochemical and/or physical processes. In particular, the reactor tank serves for providing a sterile chamber for culturing cells and/or microorganisms. In a preferred embodiment of the reactor tank according to the invention, for this purpose the bottleneck of the reactor tank is tightly closed by means of a lid, wherein the lid possesses at least passages and/or connections for the gas and liquid supply and removal for the reactor tank. According to the invention, the lid does not have a passage for a drive axle [FIGS. 2-5]. The lid is a further element of the reactor tank according to the invention. Preferably, the connected gas lines are fitted with sterile filters, wherein the sterile filter of the off-gas line is preferably fitted with a heating mat in order to keep condensate away from the filter surfaces. Alternatively, the off-gas, for avoiding condensate on the filter, can be cooled down to a lower dew point (condensation temperature <ambient temperature) with an off-gas cooler, e.g. via an electronic cooling element (e.g. a Peltier element) which is applied to a heat-transfer surface produced from film materials.

In addition, the lid, if necessary, can comprise further passages and/or connections for elements from the group comprising:
one or more electronic, optoelectronic or electrochemical sensors, in particular disposable electrochemical sensors from US 2012/0067724 A1 or PT100 resistance sensors for temperature control and/or capacitive sensors for level control or for cell density measurement,
an internal cell separator and/or
a sampling system.

The reactor tank, depending on application, is appropriately fitted with one or more of said elements.

In a preferred embodiment of the invention, the lid is composed of a stopper and a retainer sleeve. The stopper is usually produced of plastics selected from the group of polyether ether ketones, thermoplastic or silicone. Usually, the stopper is made as a disposable stopper, in a particular embodiment, alternatively, reusable.

Preferably, the stopper is introduced into the neck of the reactor for closure, sealed against the inside of the bottleneck by means of an O-ring seal mounted on the periphery, and, with a separate locking means such as, e.g., a screw-mountable retainer nut, screwed onto the thread of the bottleneck or clamped with a clamping ring. Alternatively, the stopper introduced into the bottleneck can be sealed by means of a sealing lip applied on the bottle opening and clamped with a separate screw-mountable retainer sleeve and screwed onto the plastic bottle. A further alternative is a lid which contains the identical passages as the stopper which is screwed on the plastic bottle and sealed off from the bottleneck and/or the bottle opening by an O ring. Preferably, the stopper pushed into the bottleneck is used, which is sealed with an O-ring seal on the bottleneck and which is screwed firmly with a separate screw-mountable retainer nut on the plastics bottle [FIG. 2]. This embodiment has the advantage that the O ring has little mechanical stress and no twisting of the flexible tubular line occurs, as would be the case when a lid is turned.

The reactor tank together with lid is preferably constructed as a disposable element, i.e. it is preferably intended not to clean the complete reactor tank after use, but to dispose of it. Therefore, the reactor tank preferably comprises only the essential elements which are necessary for providing a sterile reaction chamber:

The plastics bottle is usually produced and used as a disposable article.

For the culture of sensitive cells, or the production of clinical cell products, the gas is preferably supplied exclusively via the surface. In this case, the lid has no passage for a bubbling gas introduction element and the reactor tank according to the invention has no internals for bubbling gas introduction. For applications in the context of process development with the focus on scale up to large fermenters, for perfusion methods having high cell densities and for microbial processes, an installation can be provided for additional microscale or macroscale gas introduction (e.g. supplied by flexible tubular lines from the top via the lid and a sintered body adhesively applied to a container wall). Preferably, the reactor according to the invention may be produced completely from inexpensive elements and hereby permits the use of the reactor as a disposable system. Alternatively, all high-value elements are integrated into a reusable lid and only the reactor tank is used as a disposable element.

In a particular embodiment of the reactor, for cell retention a cell separator in the reactor tank is used. According to the invention, the internal cell separator is formed by a central vertical separator tube and a separator head having a collector for removing by suction culture solution freed from cells, wherein the lid has a passage for the collector and the cell separator is either rotatably mounted or statically fixed to the lid. The tube and the separator head can have differing lengths, geometry (conical and straight) and diameters, and have diverse tube internals (conical and ring internals, flow aligners). Particular embodiments are shown in FIGS. 6 to 8. The cell separator can be made of steel, glass or plastic. Preferably, it is made of plastic such as, for example, polyethylene, polypropylene, polyethylene terephthalate, polyether ketone and/or polycarbonate and used as a disposable element.

The present invention therefore further relates to an internal vertical cell separator for bioreactors formed by a central vertical separator tube and a separator head having a collector for removing cell-free medium by suction, wherein the cell separator is fixed or rotatably mounted to a lid for a reactor tank and the lid has a passage for the collector.

If a cell separator is used, the reactor tank usually has a wide neck, in order that the prefabricated cell separator fastened to the lid can be introduced through the bottleneck. If the cell separator is moveably fixed (=rotatably mounted) to the lid, it is only minimally affected by the rotary motion of the reactor tank owing to its inertia. As a result, the circulation flow transmitted by the separator into the sedimentation chamber and interfering with the sedimentation process are avoided, and the retention considerably improved, as shown in FIG. 10.

The cell separator is preferably designed in such a manner that inner and outer region of the cell separator are substantially separated from one another by corresponding constrictions. In this manner, a transmission of the flows interfering with the sedimentation from the well mixed outer chamber into the sedimentation zone are reduced. In other words, the internal volume of the cell separator shall be affected as little as possible by flows in the outer volume (=culture volume), but a back-transport of the retained cells into the mixed supplied reactor region must remain ensured.

Preferably, the internal cell separator, for a reactor tank having the dimensions of cross sectional edge length D=120 mm, H=235 mm, has a separator tube (310) having a separator length l (370) from 40 mm to 200 mm, in particular from 90 to 190 mm, preferably 190 mm (FIGS. 6 and 7). In general, a ratio 1/H from 0.2 to 0.9 is used, preferably 0.5 to 0.9, in particular 0.8.

The separator tube has a round cross section with a tube diameter d (350), wherein the ratio of tube diameter d to the bottle cross section edge length D is usually from 0.25 to 0.90, in particular from 0.5 to 0.85, preferably 0.83. The tube diameter d is of importance for the cell retention to achieve the separator surface area.

It is preferred to select the bottleneck and cell separator cross sections in such a manner that the cell separator can be readily introduced into the bottle. This is necessary, in particular, when a reusable autoclavable lid is to be used which is to be connected to the gamma-sterilized reactor tank under the cleanbench.

In a first embodiment of the bioreactor, the gas introduction proceeds solely via the surface (FIGS. 6 and 7). For this purpose the tube diameter d (350) of the separator is selected in such a manner that a ratio of the culture volume V_{K} supplied with gas via the surfaces defined by formula (I) and separator volume V_{A} defined by formula (II) is from 0.01 to 10, preferably from 0.2 to 2.

$$\begin{matrix}{V}_{K} = {D}^{2} * L - \frac{π}{4} {d}^{2} ( L - S ) & ( I ) \\ {V}_{A} = \frac{π}{4} {d}^{2} l & ( II )\end{matrix}$$

Further parameters of the cell separator are the separation area (=clarification area) A defined as

$$A = \frac{π}{4} {d}^{2} \qquad ( III )$$

and also the clarification area loading v=q/A, wherein q is the harvest stream.

At the separator head, there is situated the collector (320) for removing the cell-free culture solution by suction. Usually, the ratio dv/d of the collector diameter dv (360) to the tube diameter d is 0.1 to 0.8, preferably 0.3-0.5.

Preferably, the collector (320) has a conical shape. This shape has the advantage that more space is available for introducing further elements (sensors, sampling line, etc.) via the lid. Likewise, the gas-introduction area is more slightly reduced.

Preferably, the separator in the reactor tank is used with a ratio l/s of the separator length l to the bottom spacing s from the separation tube of 0.75 to 0.9.

Internals in the separation tube and collector (320) are preferably dispensed with in the cell separator.

In hydrodynamic studies with the model particle PAN-X, a tube statically built in at the lid having differing tube lengths geometry (conical/straight head) and diameters and also diverse tube internals (cone and ring internals, flow aligners etc.) was studied. According to the experiments available to date, the statically installed (=corotating), internal cell separator for solids in the range of clarifying area loading v of 0.025<v [m/h]<0.2 at a power input P/V_{K} of 3 W/m3 has a comparable degree of retention to static external systems (e.g. plate separator, vertical flow sedimentation tank) (FIG. 13).

In particular, the cell separator according to the invention is applicable for the culture of readily sedimentable particles such as, e.g., support-fixed cells which is useable, depending on the sink velocity of the support materials, in considerably higher power introduction ranges P/V_{K} of >>3 W/m3.

In a particular embodiment of the invention, the reactor according to the invention, in addition, has an automatic sampling element.

In a first embodiment, the sampling element consists of a receiving line which is conducted through the lid (=lid sampling element, see FIG. 10). Outside the reactor, the line can be shut off by means of clamps and pinch valves. Via an adjoining Y piece, there proceed, firstly, the vertical removal by suction of the sample from the fermenter by means of reduced pressure and the subsequent transport to the sampling preparation station by means of overpressure. This Y-shaped sampling element is particularly advantageous for achieving an automatic sampling module consisting of flexible tubular lines, pinch valves, sterile filters and an overpressure supply and a reduced pressure supply. Usually, commercially available lines, valves and Y pieces made of plastic are used, so the sampling element that can be integrated into the lid can be provided and used as a disposable element. The basic principle of the Y-shaped sampling element is described in WO 2007/121887, and is incorporated by reference, in which two burettes are actuated in order to ensure transport and aliquoting of a sample.

After the sample removal, the sampling element is sterilized with EtOH and dried. Preferably, filter elements for air and EtOH are built in in order to prevent contamination of the sample withdrawal element. Preferably, the sampling element is coupled to the BayChromat-Platform for automated analysis from Bayer Technology Services GmbH.

In a further embodiment, the reactor tank has, on a bottle wall, in particular on the wall opposite the sensors (sensor patch or electrochemical sensors), a passage and/or a connection in the region close to the bottom for mounting a sampling system. Examples of passages and/or insertions are, inter alia, standardized Ingold stubs—or PG13,5-thread stubs. A suitable sampling system is described, e.g., in DE102008033286 A1.

The mixing within the reactor tank according to the invention proceeds via the reactor tank rotation changing direction periodically, which in combination with the angular shape of the plastics bottle, causes inwardly directed wave-shaped flows to the surface of the reactor contents. For configuration of the reactor motion, the teaching of WO 2010/127689 is incorporated by reference.

All remaining elements which are required for operating a reactor, in particular for culturing cells and/or microorganisms, in particular a drive unit for generating the reactor tank rotation changing direction periodically and optoelectronic sensor system for reading the sensor patches are provided by peripherals and are reusable. The reactor, which in the prior art is usually one coherent unit, is therefore in the present case preferably divided into separate parts which are configured according to their functions.

A further element of the reactor according to the invention is therefore the peripherals. In particular, as peripherals, reactor tank receiving peripherals are used which have one or more reactor tank holders, wherein the reactor tank and the reactor tank holder are, as separate parts of an overall system, matched to one another in such a manner that the reactor tank can be introduced into the reactor tank holder and/or in particular clamped in there, and is supported thereby in the liquid-filled state.

The reactor tank receiving peripherals for receiving a reactor tank according to the invention is a further element of the reactor according to the invention and comprises at least:
one or more reactor tank holders for receiving one reactor tank in each case comprising the footprint adapted to the reactor tank and one or more lateral fixing elements. For example, the reactor tank holder has a fixing plate and lateral clamping arms or clamping surfaces.
A drive unit for carrying out a rotary motion which changes direction periodically, such as a stepper motor, for example, is connected to the reactor tank holders, in particular to the fixing plate of the reactor tank holders. Preferably, a stepper motor without gearing with direct coupling of the motor and the drive is used. By way of the drive unit, the reactor tank can be put into a rotary motion changing direction periodically about its fixed vertical axis, in such a manner that direct coupling of the drive unit to the reactor tank itself is not required. Preferably, for implementing the reactor motion, a stepper motor without gear is used. Such an arrangement has the advantage of being very low noise. Preferably, the drive unit is controllable by means of a control unit. Usually, the control is part of the drive unit.
One or more optoelectronic sensor systems installed on the reactor tank holder, in particular on one of the lateral fastening elements, for reading the sensor patches mounted at the side at a position of the reactor tank defined by coordinates, in particular a pH transmitter and/or an oxygen content transmitter. By way of the direct coupling of the light excitation and detection units to the sensors, the light intensities for generating evaluatable measurement signals and therefore generating radicals may be considerably decreased, which proves advantageous for a prolonged service life of the sensor patches.

According to the invention, the data transmission proceeds in a line-bound manner via differential serial interfaces and/or wirelessly by radio such as, for example, Bluetooth of WLAN. Preferably, the optoelectronic sensor system possesses the differential serial interface for symmetrical signal transfer of the EIA485/RS485 type, on account of the robust data transmission and high tolerance to electromagnetic interference. For improved data transmission, a stepper motor without gear with direct coupling of the motor and the drive was identified as particularly advantageous, because it permits a particularly interference-free data transmission.

FIG. 11 shows a particular embodiment of the reactor including reactor tank receiving peripherals.

Preferably, the footprint adapted to the reactor is exchangeable or adaptable in such a manner that the reactor frame is applicable to reactors of various sizes.

The present invention also relates to the use of the reactor according to the invention and reactor tank and also to a method for the culture of cells and/or microorganisms.

In the reactor tank, during operations, the ratio of liquid level to reactor tank width is preferably 0.05 to 2, and particularly preferably 0.1 to 1, wherein the liquid level can change as a consequence of supplemental feeding with the growth of the cells. In addition, the reactor tank, while maintaining the preferred hydrodynamic and processing properties thereof, is operated with a sufficient head space between reactor tank head and liquid level (=full level 180, H_{L}) of at least 5% to 50% liquid height, preferably at least 25% liquid height, in order, in the case of foam formation, to ensure sufficient spacing to the gas discharge line equipped with a sterile filter. For control of the fill level, usually, a capacitive sensor for fill level control is used through the lid or on one of the container walls.

It has surprisingly been found that a comparatively small angular amplitude is sufficient for the rotating-oscillating motion of the reactor in order to achieve good mixing and/or sufficient intensification of transport processes. In particular, it is scarcely necessary to achieve 3600° rotations (that is equivalent to 10 rotations) of the reactor, and so there is no requirement for structurally complex solutions for linking the oscillating and rotating reactor to the static surroundings (e.g. for feeding and removing media and gases, electrical energy and electrical signals).

In the use according to the invention, the reactor tank is moved at an angular amplitude α in the range from 2°≦|α|≦3600°, preferably 20°≦|α|≦180°, particularly preferably 45°≦|α|≦90° in a rotating-oscillating manner, wherein deviations from ±5° may be present. In particular, |α|=60° is considered to be very particularly preferably in the use of particularly low-shear bioreactors supplied with gas via the surface. In total, therefore, the oscillating motion sweeps through an angle of 2|α|.

Experiments have found that when the power input is elevated, movement states can be established in the reactor in which gas bubbles are introduced into the reactor medium. Gas bubbles are drawn in from a power input of P/V_{K}>10 W/m³. For cells and/or microorganisms which are not damaged by bubbling gas introduction, a very simple increase in gas supply can be achieved in this manner. Via an additional bubbling gas introduction via a sintered tube preferably installed in the bottom region, the mass transport can be considerably improved. The flow generated by the rotary oscillation ensures gentle detachment of the microbubbles from the sparger and thus a large phase interface a or a large mass transfer coefficient k_{L}a.

The invention will be described in more detail hereinafter with reference to figures, but without restricting it to the embodiments shown.

### DESCRIPTION OF THE FIGURES

FIG. 1 shows schematically the side longitudinal section of a preferred embodiment of the reactor tank according to the invention and reactor tank holder in side view. FIG. 2 shows a schematic view of a stopper thermoplastic design from the top.

FIG. 3 shows a schematic section of a stopper silicone design having hose nozzles (135) fastened with a retaining sleeve (120) and an O-ring seal (140).

FIG. 4 shows schematically in front view a section through a stopper silicone design fastened with retaining sleeve (120) and sealed by a sealing lip (140b).

FIG. 5 shows schematically in front view a section through a screw-mountable plastic lid with hose nozzles (135).

FIG. 6 shows schematically in front view a straight tubular separator having a straight top (=abrupt cross sectional constriction) in longitudinal section.

FIG. 7 shows schematically in plan view a straight tubular separator having a straight top.

FIG. 8 shows schematically in front view a straight tubular separator having a straight top (=abrupt sectional constriction) and flow inverter.

FIG. 9 shows schematically the experimental structure including lines, wherein a reactor having a straight tubular separator which has a conical head and is statically fastened in the lid is shown by way of example.

FIG. 10 shows the schematic structure of an automated lid sampling element having a sample intake line (1110) conducted through the lid, which sample intake line is connected via a Y piece (1170) with a sample line (1120) to an automated platform (1190) for fully automated withdrawal and plug-type transport of liquids and also to further lines for air feed and ETOH cleaning and sterilization (1210).

FIG. 11 shows a particular embodiment of the reactor tank together with reactor tank receiving peripherals.

FIG. 12 shows experimental results R=f(PN) in the comparison of the static and co-rotating separator according to FIGS. 6 and 7 and verifies the highly surprising necessity of a rotatably mounted sedimentation tube which, in contrast to the co-rotating variant, even at relatively high power inputs PN_{K}>3 W/m3 ensures good particle retention.

FIG. 13 shows comparative experiments regarding other sedimentation separators.

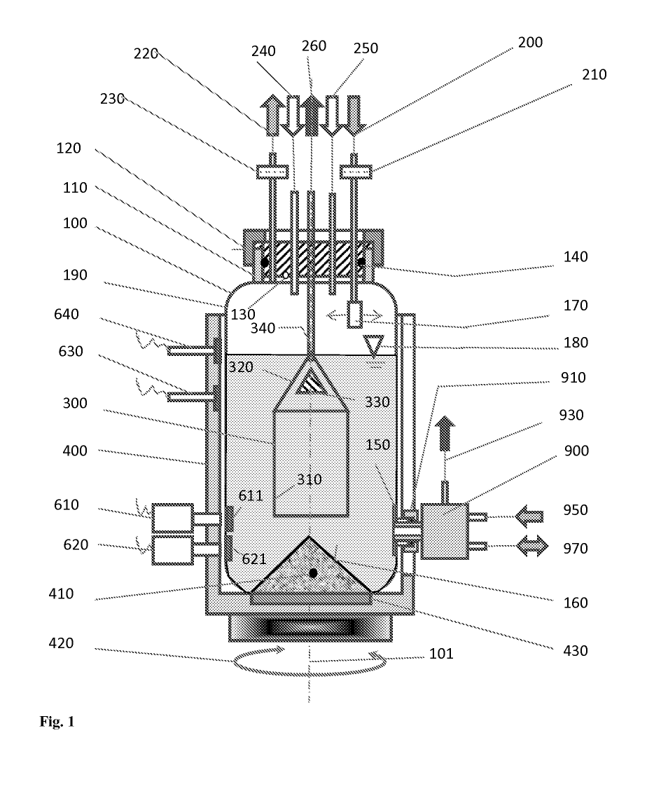

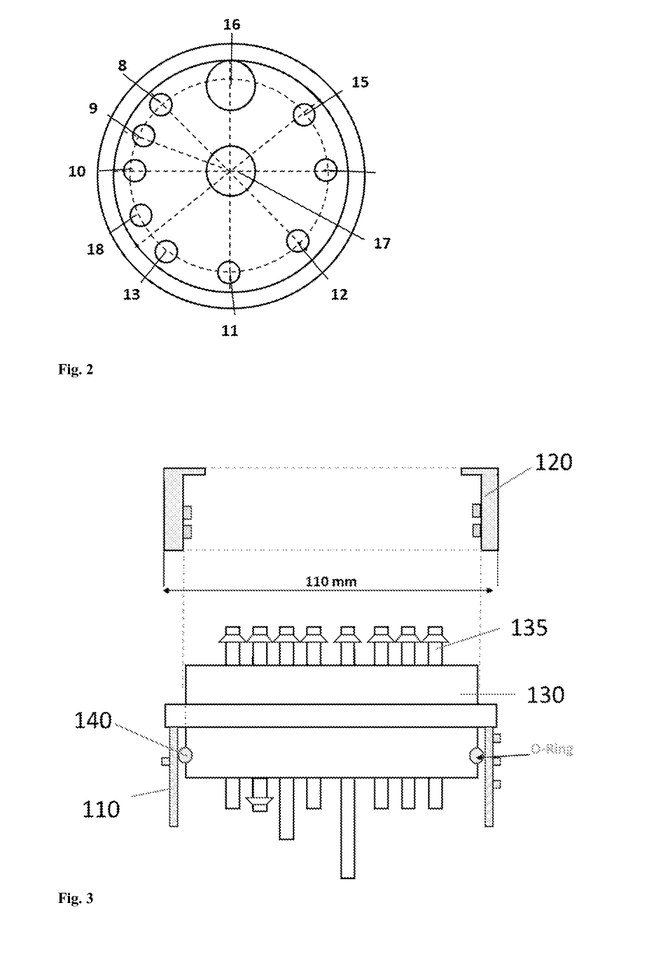

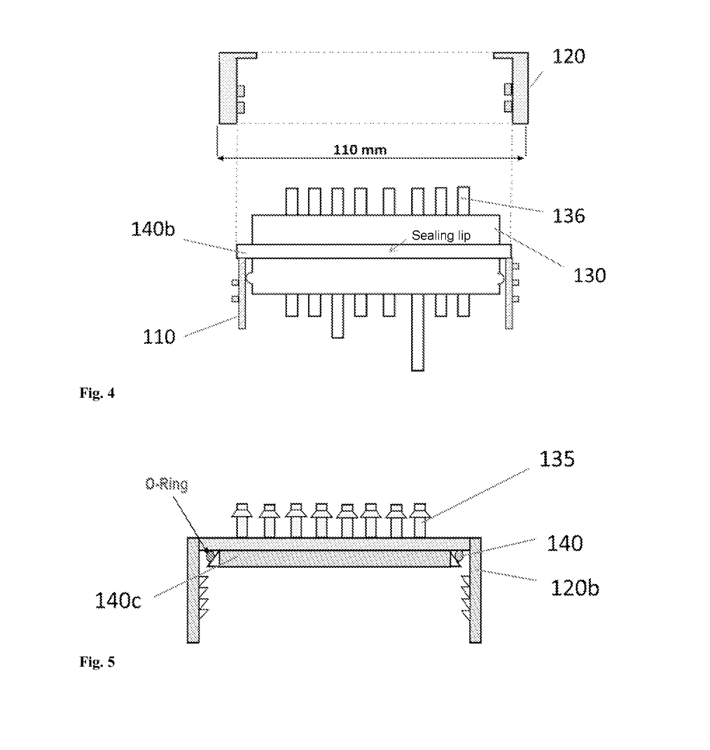

### REFERENCE SIGNS

8, 9 Port (ID 2 mm, OD 3 mm, port length: 15 mm)
10 to 15 Port (ID 3 mm, OD 4 mm, port length: 15 mm, port 10 with hose nozzle on both sides)
16, 17 PG13.5 Port
18 Identical port type to PG13,5 with ID 4 mm
100 Container
101 Axes of rotation
110 Bottleneck
120 Retainer nut/retainer sleeve
120b Screw-mountable lid
130 Stopper
135 Hose nozzle
136 Silicone tube
140 O-Ring
140b Sealing lip
140c Seal
150 Stub
160 Bottom
170 Gas distributor
180 Fill level H_{L}
185 Bottle height H
186 Bottle cross section D
190 Bottle wall
198 Culture volume V_{B}
200 Gas feed
210 Sterile filter
220 Gas removal
230 Sterile filter
240 Medium
250 Additives
260 Harvest
261 PG13.5 stub (lid introduction)
270 Filter heater (19 Jan 12)
280 Side sampling (19 Jan 12)
290 Sensor port (chem.) (19 Jan 12)
300 Settler cell separator
310 Settler tube/cylinder
320 Collector/cone
330 Collector fitting/flow inverter
331 Opening
332 Gap
333 Sediment takeoff
340 Outlet/takeoff tube
350 Tube diameter d
360 Collector diameter dᵥ
370 Separator length l
380 Distance from bottom s
390 Fill level
395 Separator volume V_{A}
400 Holder
410 Heat conducting element
420 Oscillation
430 Heating mat
610 pH measurement
611 pH spot
620 pO₂ measurement
621 pO₂ spot
630 Temperature measurement
640 Fill level measurement
700 Drive unit with motor
710 Opto-electronic measurement system
720 Control cabinet
730 Temperature measurement
740 Rotary knob
750 Display
760 Reactor frame
900 Sample valve
910 Retainer nut
911 Seal
912 Tube
921 Nonreturn valve
922 Nonreturn valve
923 Membrane
930 Sample line
931 Sample
950 Purge line
951 Sterile filter
952 Gas
953 Steam
960 Sample distribution
970 Pressure line
971 Reduced pressure
972 Overpressure
980 pH measurement
981 pH sensor
982 Buffer
983 Waste
990 Coupler (Luer-Lock)
1100 Lid sampling element
1110 Sample intake line
1120 Sample line
1130 Clamp
1140 Liquid filter
1150 Air filter
1160 Pressure reducer
1170 Y Piece
1180 Pinch valves
1190 Automated platform for fully automated withdrawal of liquids, transport, sample preparation and subsequent analysis as known, e.g., from EP-1439472A1 and EP-2013328A2 (BaychroMAT®)
1200 Feed of cleaning solution
1210 Air feed

### EXAMPLE

### Bioreactor Bottle

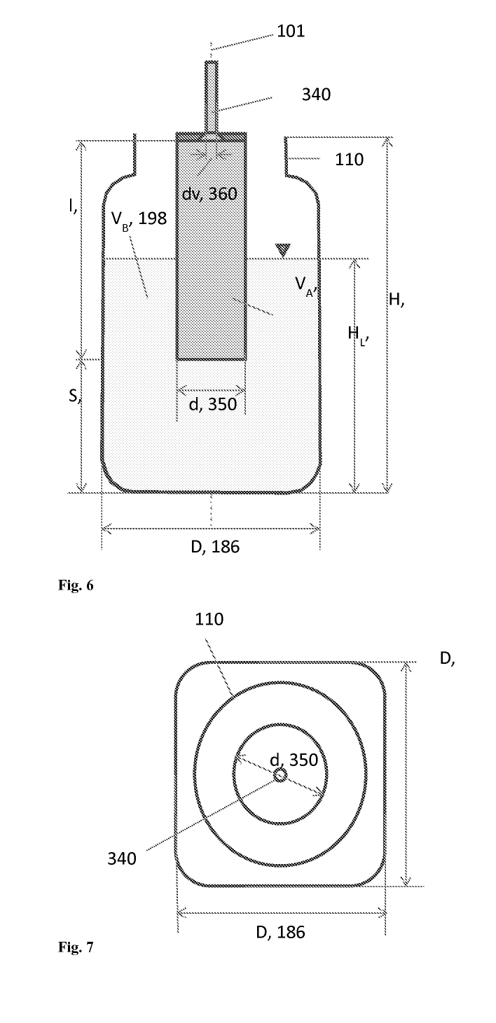

A disposable plastics bottle having a square cross section and cross section edge length D=120 mm, a height H=235 mm and a round bottleneck 110 having a neck cross sectional diameter of 105 mm served as container 100. The container had rounded edges (FIGS. 6 and 7); however, this scarcely affected the characteristics of the system. The drive was performed using a stepper motor which acted directly on the bottle holder (FIG. 11).

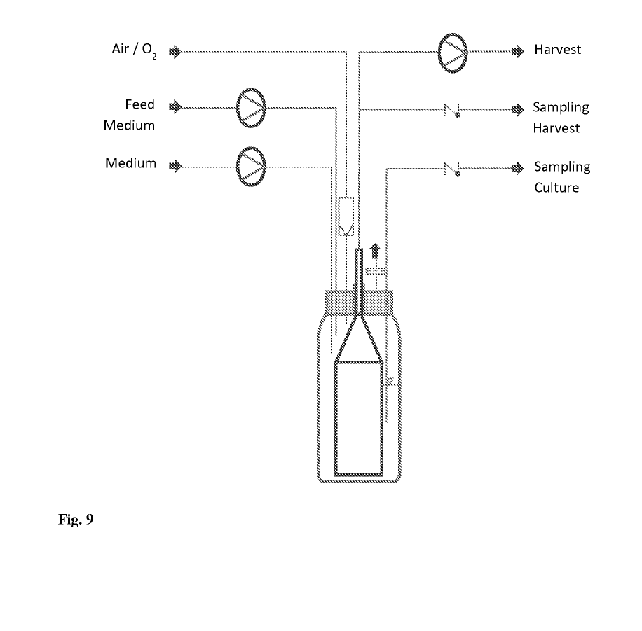

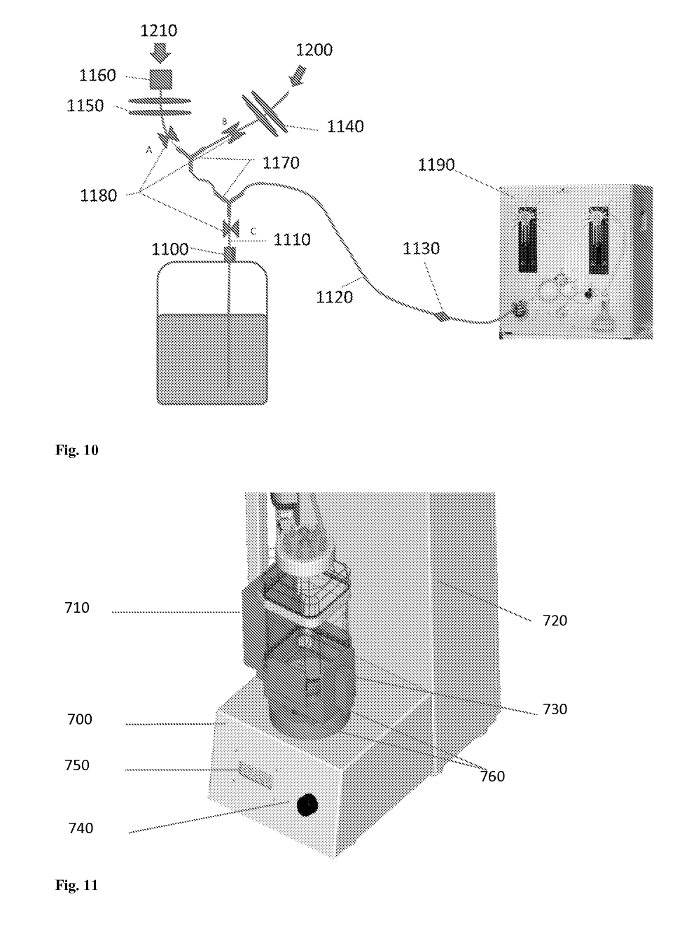

A cell separator 300 was built into the container 100 in order to operate the bioreactor as a perfusion system (FIG. 9). The cell separator 300 comprised a suction tube 340 conducted through the lid 120b vertically connected to a cylindrical separator tube 310 having a tube diameter d=350) of 70 mm, wherein the transition region between the suction tube 340 and the upper region of the separator tube 310 formed the harvest stream collector 320 and the separator volume V_{A} formed the lower part open at the bottom of the separator tube 310 (FIG. 7).

In order to dispense with moving seals, the suction tube 340 was integrated fixed into the lid 120b and therefore followed the rotary motion changing direction periodically (also termed oscillation movement) around the fixed axis (101) of the bioreactor (corotating embodiment). For comparative experiments, alternatively, the suction tube 340 was fastened to a stand; in these experiments, the cell separator 300 was then used statically.

In the perfusion operation, the separator tube 310 projected into the suspension situated in the vessel (degree of filling 390> spacing from the bottom s, 380).

By way of a perfusion pump (peristaltic pump from Watson & Marlow) attached at the harvest stream collector 320, the suspension was withdrawn by suction from the bottom into the separator volume V_{A} of the separator tube 310. Within the separator tube 310 the suspension ascended and was clarified by sedimentation of the cells/particles (vertical separation). The particles fell against the direction of flow downwards out of the separator volume back into the culture volume V_{K} (FIG. 7). The clarified solution was collected from the harvest stream collector 320 of the separator tube 310 and removed via the suction tube 340.

The clarifying area A of the separator tube corresponds to the circular cross section thereof and is calculated according to Equation III.

### PAN-X Studies:

The particle system PAN-X (polyacrylonitrile, spherical particles from Dralon GmbH) was used as model particle for study of separation performance of the reactor bottle according to the invention with integrated cell separator in cell culture.

For examining the identity of the physical properties, the particle size distribution and the particle falling velocity were compared, since they are the determining factors of sedimentation.

The particle size distribution was determined via laser diffraction method (Mastersizer 2000, measured according to the operating instructions). The results were plotted as particle volume in % based on the total volume, as a function against particle size in μm. The modal value X_{Mod}, states what particle size is most frequently represented in volume terms and was approximately 21 μm.

The falling velocity was analysed using a sedimentation balance. For this purpose a suspension was produced which has the same concentration as that used in the experiment. The PAN-X was suspended in desalinated water (=completely ion-free (CIF) water) and had a mass concentration of approximately 3 g/l or a volume concentration of 0.88 by volume. A temperature of 20° C. was selected for the analysis. Falling velocities vₛ, of 0.129 m/h to 0.137 m/h measured under the experimental conditions were determined in various PAN-X batches and correspond to the conditions of non-hindered sedimentation.

CHO cells have, for example, a sedimentation rate of 0.0145 m/h [Searles J A, Todd P, Kompala D S, Biotechnol Prog (1994) 10: 198-206] and are thus relatively slowly sedimenting cells. The hybridoma cell line AB2-143.2 has a sedimentation rate of 0.029 m/h [Wang Z, Belovich J M (2010) Biotechnol Prog 26 (5): 1361-1366].

For production of the model suspension, 3 g of PAN-X were weighed out and suspended in 1000 ml of CIF water, using a magnetic stirrer. For sampling, the harvest stream was collected in a measuring cylinder, while the volume taken off was replaced by CIF water up to a fill level H/D=1 by means of a second peristaltic pump.

Where not stated otherwise, all experiments were carried out with the following standard parameters:
Acceleration a=1000°/s² (PN=11.12 W/m³)
Corotating separator
Clarification area loading v=0.1 m/h
Distance from the bottom s=70 mm

Gravimetric determination of the particle concentration: the particle concentration in the harvest stream was determined gravimetrically by filtering off (filtration by suction) a defined volume of harvest stream and subsequently drying and weighing the filter by means of a drying balance.

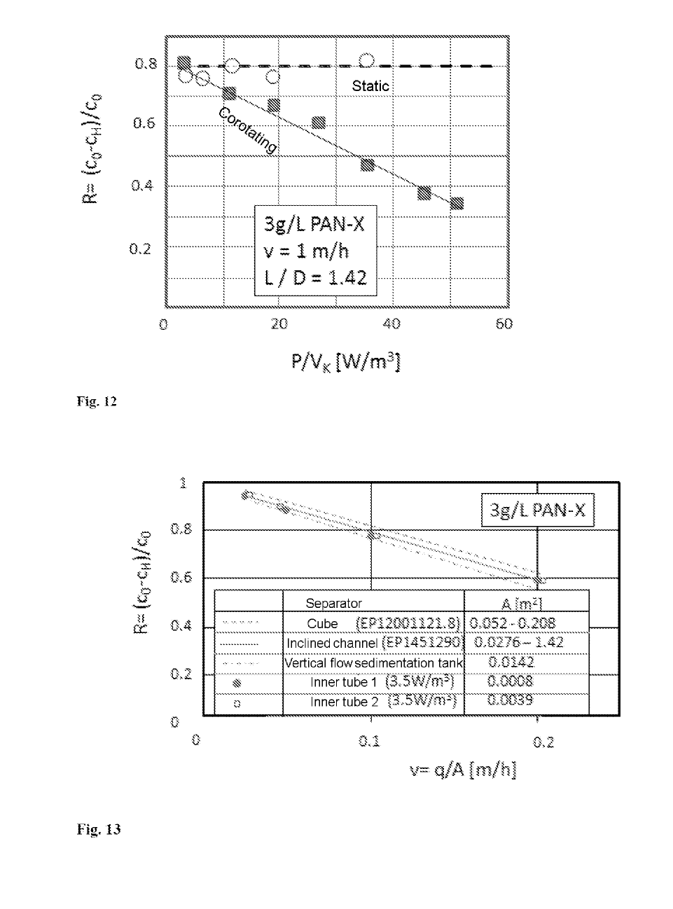

The effect of acceleration or power input on the degree of retention at various separator lengths 1 (=370) was determined. For this purpose, experiments with a static separator and corotating separator were compared (for results see FIG. 12). The harvest stream collector having an abrupt cross sectional constriction according to FIGS. 6 and 7 was installed on a separator tube having a diameter d=70 mm. Length L of the separator of 90 mm and 170 mm at accelerations of 600 to 2000°/s² were studied.

Comparison of performance in the retention of the PAN model particles at various power inputs P/V of up to P/V=50 W/m³ and clarification area loading v=0.1 m/h showed that the degree of retention R decreased with increasing power input into the bioreactor with a corotating installation of the separator tube, with the degree of retention being considerably beneficially affected by increased length L of the separator.

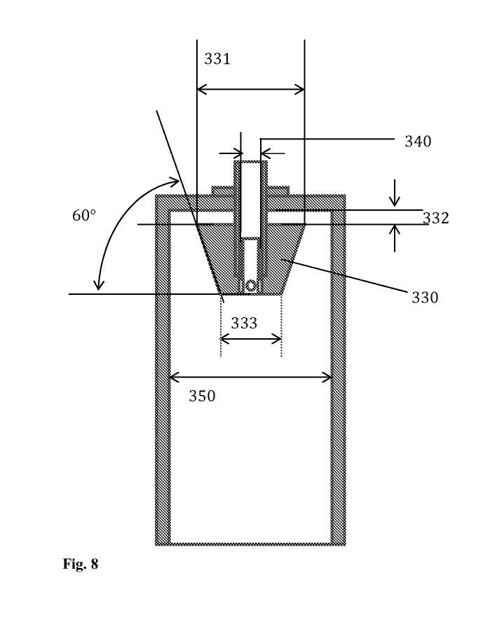

In further experiments, the effect of various harvest stream collectors was studied for a separator length 1=108 mm and a power input of PN=11.12 W/m³ (a=1000°/s²) and v=0.1 m/h. The harvest stream collector with abrupt cross sectional constriction according to FIGS. 6 and 7 having the diameter ratio dᵥ/d=5/70 (also termed more simply collector) of a harvest stream collector having a conical cross sectional constriction according to FIG. 8 having an angle of aperture of 54° and a separator length 1=58 mm were built into the lid 120b of the bioreactor so as to be corotating. The results showed the clear advantage of the conical harvest stream collector. The retention performance of this separator was surprisingly virtually constant at separator length l from 90 to 143 mm in the total experimental range. In the harvest stream collector having an abrupt expansion, the separation performance increased with increasing separator length l from 90 to 170 mm, but without achieving fully the performance of the conical cross sectional constriction.

Effect of the Distance from the Bottom:

For investigation of the distance from the bottom, a separator tube having a simple harvest stream collector according to FIGS. 6 and 7 and separator length 1=90 mm was used. It was adjusted to have distances from the bottom s from 10 to 90 mm. Under standard experimental conditions of P/V=11 W/m³ and v=0.1 m/h, no effect of the distance from the bottom was able to be observed with the separator tube d=70 mm and l=90 mm in the region of distances from the bottom from 10<s [mm]<70. Not until above s>70 mm is an effect of distance from the bottom visible.

### Effect of Clarification Area Loading:

The clarification area loading v of the separator tube corresponds to the velocity of the vertically ascending medium and, according to Equation IV, has a direct effect on the particle retention. The effect of the clarification area loading was studied on the separator tube according to FIGS. 6 and 7 (d=70 mm, 1=170 mm) The separator tube was fixed into the bottle lid so as to be corotating with a distance from the bottom s=70 mm. The reactor was operated at a power input of 3.43 W/m³ (a=600°/s²) and a clarification area loading from v=0.025 m/h to v=0.2 m/h. In the loading range studied from 0.025<v [m/h]<0.2, the degree of retention R decreases virtually linearly with increasing area loading or ascension rate v of the separator. This result is not only due to the characteristics of the separator, but also due to the sedimentation properties of the model suspension used. PAN-X particles are available as a polydisperse suspension having a broadly distributed particle size and falling rate. It is therefore to be assumed that the retention characteristics in the virtually monodisperse cell suspensions have a course shifted to a steeper and smaller ascension rates.

A comparison of the performance of various separation systems is shown in FIG. 13 in the form of the degrees of retention R against the clarifying area loading. Two vertical separators installed into the reactor tank so as to be corotating at power inputs of P/V_{K}≈3 W/m³ with separator areas of 8 cm² and 39 cm² (inner tubes 1 and 2) were compared with the static external gravity separator variants such as the classic vertical separation tank, an inclined channel separator according to EP1451290 and a cubic separator according to EP12001121.8. The experimental results verify a virtually equivalent retention performance of all separators over the entire ranges of clarification area loading studied from 0.025 to 0.2 m/h with slight advantages for the cubic separator.

The studies which led to this invention were promoted according to the financial aid agreement “Bio.NRW: ProCell—Innovative platform technologies for integrated process development with cell cultures” in the context of the European Fund for Regional Development (EFRD).

1. A dimensionally stable, angular plastic bottle comprising, a bottom, walls, an interior and at least one axis of the interior comprises a bioreactor tank for the culture of cells.

2. A reactor tank comprising a dimensionally stable square plastic bottle having a bottom, walls, an interior and at least one closable access to the interior, wherein the closable access consists of at least one selected from at least one bottleneck and at least one passage, and wherein one or more sensor patches are mounted in the interior, on one or more walls in the lower region of the plastics bottle, at a site defined by coordinates.

3. A reactor tank comprising a dimensionally stable angular plastic bottle having a bottom, walls, an interior and at least one closable access, wherein the closable access consists of at least one bottleneck, and wherein the bottleneck is closable by a lid, wherein the lid has at least one selected from passages and connections for the gas and liquid supply and removal into and out of the reactor tank, and no passage for a drive axle.

4. The reactor tank according to claim 3, wherein, for the gas removal, a gas discharge line equipped with a sterile filter having a heating mat is conducted through the lid.

5. The reactor tank according to claim 3, wherein one or more electronic, optoelectronic or electrochemical sensors are conducted through the lid.

6. The reactor tank according to claim 3, further comprising an internal vertical cell separator, wherein the internal vertical cell separator comprises:
a. a central vertical separator tube having a round cross section with a tubular diameter d, wherein the ratio of tubular diameter d to bottle cross section edge length D is from 0.25 to 0.90, and
b. a separator head having a collector for removing by suction culture solution freed from cells, wherein the cell separator is fixed to the lid of the reactor tank statically, or mounted so as to be able to rotate, and the collector is conducted through the lid.

7. The reactor tank according to claim, further comprising a disposable sampling element, wherein the disposable sampling element has a sampling suction line installed on the wall or on the lid of the reactor tank and connected via a Y-piece to a sample transport line, with which the sampling line is connected to an automated platform for fully automated sterile withdrawal of liquids, wherein the sample transport is supported by means of an air feed connected to the Y piece.

8. A reactor comprising:
a reactor tank formed by a dimensionally stable angular plastic bottle having a bottom, walls, an interior and at least one closable access to the interior, wherein the closable access consists of at least one selected from at least one bottleneck and at least one passage, and
comprises reactor tank receiving peripherals for receiving the reactor tank, wherein the reactor tank receiving peripherals consist of:
at least one reactor tank holder for receiving in each case one reactor tank having a footprint adapted to the reactor tank and one or more lateral fastening elements,
a drive unit connected to the reactor tank holder for carrying out a rotary motion changing direction periodically.

9. The reactor according to claim 8, wherein one or more sensor patches are mounted on one or more walls in the lower region of the plastics bottle, at a site defined by coordinates, and wherein, on the reactor tank holder, one or more optoelectronic sensor systems for excitation and reading of the sensor patches are fixed directly or by means of optical waveguides.

10. The reactor according to claim 9, wherein the one or more optoelectronic sensor systems transmit collected data in a hard-wired manner via at least one selected from differential serial interfaces and wirelessly via radio.

11. The reactor according to claim 10, wherein the drive unit is a stepping motor without gearing having direct coupling of a motor and a drive.

12. The reactor according to claim 8, wherein the closable access of the reactor tank is a bottleneck and is closable by a lid, the at least one selected from passages and connections for the gas and liquid supply of the reactor tank does not have a passage for a drive axle.

13. A reactor tank receiving peripherals for receiving a reactor tank, wherein the reactor tank receiving peripherals comprise:
at least one reactor tank holder for receiving in each case one reactor tank having a footprint adapted to the reactor tank and one or more lateral fastening elements,
one or more optoelectronic sensor systems fastened to the reactor tank holder for reading sensor patches mounted laterally on a position of the reactor tank defined by coordinates,
a drive unit connected to the reactor tank holder for carrying out a rotary motion changing direction periodically, wherein the drive unit is a stepper motor without gears having direct coupling of the motor and the drive.

14. A method for the culture of cells in the reactor according to claim 8, wherein the reactor tank is put into a rotary motion changing direction periodically having an angular amplitude α in the range from 2°≦|α|≦3600° having a power introduction >10 W/m³ around its stationary vertical axis.

15. The method according to claim 14 for culture of cells, further comprising a falling velocity of 0.01 to 100 cm/h, wherein the reactor tank as an internal vertical cell separator which is formed by a central vertical separator tube and a separator head having a collector for removing cell-free medium by suction, wherein the cell separator is fastened on a lid for a reactor tank statically or rotatably mounted and the collector is conducted through the lid, wherein a culture solution which is separated off from cells using the cell separator is removed from the reactor tank by suction.
